(19) United States
(12) Patent Application Publication
(10) Pub. No.: US 2025/0235088 A1
(43) Pub. Date: Jul. 24, 2025

(54) TOOL DRIVE ADAPTOR FOR ROBOTIC SURGICAL INSTRUMENT

(71) Applicant: Verb Surgical Inc., Santa Clara, CA (US)

(72) Inventors: Richard Lum, San Jose, CA (US); Pratik Nooji, Fremont, CA (US); Pejman Seyedi, San Jose, CA (US); Andrew Berkowitz, Redwood City, CA (US); Andrew Groth Morris, San Francisco, CA (US); Sascha Sobolewski, Santa Clara, CA (US)

(73) Assignee: Verb Surgical Inc., Santa Clara, CA (US)

(21) Appl. No.: 18/421,811

(22) Filed: Jan. 24, 2024

(51) Int. Cl. A61B 1/00; A61B 1/05; A61B 1/07; A61B 1/313; A61B 34/30; A61B 90/94

(52) U.S. Cl. CPC A61B 1/00149; A61B 1/00114; A61B 1/05; A61B 1/07; A61B 1/3132; A61B 34/30; A61B 90/94; A61B 2034/302

(57) ABSTRACT

Systems and tool drive adaptors for actuating surgical instruments with a robotic surgical system. A device for controlling rotational movement of a surgical instrument includes a rotor gear attached to a shaft of the surgical instrument. The device further includes a first helical gear rotatably coupled to the rotor gear and first drive shaft disposed through a bore of the first helical gear. The device further includes a second helical gear rotatably coupled to the rotor gear and a second drive shaft disposed through a bore of the second helical gear.

### TECHNICAL FIELD

This disclosure is directed to mechanical and electronic adaptors and more specifically to drive adaptors for robotic surgical instruments.

### BACKGROUND

Endoscopic surgical instruments are often preferred over traditional open surgical devices because the small incision tends to reduce post-operative recovery time and associated complications. In some instances of endoscopic visualization, it is desirable to manipulate the endoscope or other tool with a robotic actuator. Robotic surgery, also known as robot-assisted surgery, is a form of minimally invasive surgery that utilizes robotic systems to assist surgeons in performing precise and complex procedures.

Robotic surgery can provide enhanced precision and dexterity. In some cases, a robotic system is capable of greater range of motion and finer control than a human surgeon, and this allows the human to utilize the robotic system to perform intricate procedures with enhanced precision. Robotic surgical systems may also provide improved visualization of a scene, and the robotic manipulations may enable a surgeon to view the scene from varying angles and vantage points.

However, robotic surgical systems known in the art fail to provide precise rotational control of rigid tools utilized in minimally invasive surgery, such as endoscopes, retractors, forceps, clamps, suction tubes, staplers, and so forth. In many cases, it is necessary to rotate a surgical tool clockwise or counterclockwise throughout a surgery. Thus, what is needed are systems for mechanical and electronic communication between a surgical tool and a robotic surgical system that enable precise rotational control of the surgical tool.

In view of the foregoing, disclosed herein are systems, methods, and devices for tool drive adaptors for robotic surgical systems.

### BRIEF DESCRIPTIONS OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1 is a schematic block diagram of components of a robotic surgical system;

FIG. 2 is an exploded perspective view of a portion of a surgical instrument comprising a tool drive adaptor for facilitating mechanical and electronic communication between the surgical instrument and a robotic surgical system;

FIG. 3 is an exploded perspective view of a portion of a surgical instrument comprising a tool drive adaptor for facilitating mechanical and electronic communication between the surgical instrument and a robotic surgical system;

FIG. 4 is an exploded perspective view of a portion of a surgical instrument comprising a tool drive adaptor for facilitating mechanical and electronic communication between the surgical instrument and a robotic surgical system;

FIG. 5A is a perspective view of a portion of a surgical instrument comprising a tool drive adaptor for facilitating mechanical and electronic communication between the surgical instrument and a robotic surgical system;

FIG. 5B is a perspective view of a portion of a surgical instrument comprising a tool drive adaptor, wherein the tool drive adaptor is hovering over a corresponding tool receptacle of a robotic surgical system;

FIG. 6 is a cross-sectional perspective view of a portion of a surgical instrument comprising a tool drive adaptor, wherein the tool drive adaptor is hovering over a corresponding tool receptacle of a robotic surgical system;

FIG. 7 is a perspective view showing an underside of a housing of a tool drive adaptor for a surgical instrument;

FIG. 8 is a perspective view showing an upper side of the housing of a tool drive adaptor for a surgical instrument;

FIG. 9 is a perspective view of a gear assembly of a tool drive adaptor for a surgical instrument;

FIG. 10 is a cross-sectional view of a gear assembly of a tool drive adaptor for a surgical instrument;

FIG. 11A is a schematic illustration of an example system for endoscopic visualization with color imaging and advanced imaging;

FIG. 11B is a schematic illustration of an example image pickup portion of a system for endoscopic visualization with color imaging and advanced imaging;

FIG. 11C is a schematic illustration of an example emitter and controller of a system for endoscopic visualization with color imaging and advanced imaging; and

FIG. 12 is a schematic block diagram illustrating example components of a computer system, such as components of a robotic surgical system.

### DETAILED DESCRIPTION

Disclosed herein are systems, methods, and devices for facilitating mechanical and electronic communication between a surgical tool and a robotic surgical system. The tool drive adaptor components described herein may be implemented with any suitable surgical tools that will be rotated clockwise or counterclockwise during use. Such surgical tools may specifically include endoscopic instruments, such as endoscopic cameras, retractors, cauterizers, staplers, cutters, clips, clamps, scissors, and so forth. The tool drive adaptor described herein may be implemented for rotational control of any tool utilized in minimally invasive surgical systems.

The tool drive adaptor described herein is configured to interface with a tool receptacle of a robotic surgical system. The tool drive adaptor is specifically implemented to enable the robotic surgical system to control rotational positioning of a surgical instrument and to cause clockwise or counterclockwise rotation of the surgical instrument in real-time. The robotic surgical system includes motors for driving gears within the tool drive adaptor, and the rotation of these gears ultimately causes rotation of a component of the surgical instrument. In some cases, the tool drive adaptor is implemented to rotate a rigid surgical instrument such as a laparoscopic camera, retractor, cauterizer, stapler, cutter, clamp, and so forth.

A tool drive adaptor described herein includes at least two helical gears that are each rotationally coupled to a rotor gear and an axis located between the rotor gear and the helical gear. The rotor gear is attached to a shaft of a surgical instrument such that clockwise rotation of the rotor gear causes clockwise rotation of the shaft, and vice versa. The at least two helical gears are synchronously rotated and simultaneously engage with the rotor gear. This increases the amount of driving force applied to the rotor gear, and thus enables the helical gears to rotate a relatively heavy or cumbersome device, such as a shaft of a laparoscopic surgical instrument.

Further described herein are systems, methods, and devices for digital visualization that may be primarily suited to medical applications such as medical endoscopic imaging. An embodiment of the disclosure is an endoscopic visualization system that includes an emitter, an image sensor, and a controller. The emitter includes a plurality of separate and independently actuatable sources of electromagnetic radiation (“EMR”) that may be separately cycled on and off to illuminate a scene with pulses of EMR. The image sensor accumulates EMR and reads out data for generating a plurality of data frames. The controller synchronizes operations of the emitter and the image sensor to output a desired visualization scheme based on user input. The visualization scheme may include a selection of one or more of color imaging, multispectral imaging, fluorescence imaging, topographical mapping, or anatomical measurement.

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the structure, systems, and methods are disclosed and described, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element or step not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

As used herein, the term “proximal” shall refer broadly to the concept of a portion nearest an origin.

As used herein, the term “distal” shall generally refer to the opposite of proximal, and thus to the concept of a portion farther from an origin, or a farthest portion, depending upon the context.

As used herein, the terms “elliptical” or “ellipse” refer to a plane curve surrounding two focal points such that for all points on the curve, the sum of the two distances to the focal points is a constant. As described herein an “elliptical” geometry shall include a circular geometry.

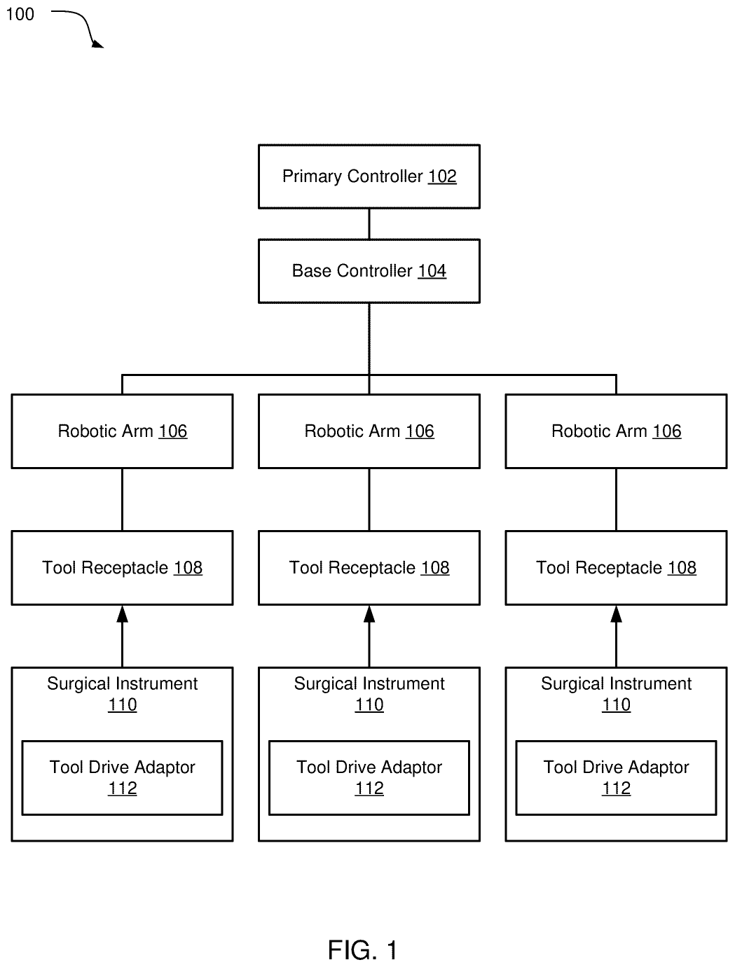

Referring now to the figures, FIG. 1 is a schematic block diagram illustrating some components of a system 100 for robotic surgery or robot-assisted surgery. The system 100 includes robotic components and surgical instruments to assist surgeons in performing complex surgical procedures with enhanced precision and control. The system 100 is typically affixed with robotic arms equipped with surgical instruments, and these robotic arms are capable of providing increased precision and dexterity than human hands, which enables surgeons to perform intricate maneuvers with improved accuracy. The system 100 is also equipped with complex visualization systems, which are described herein. These visualization systems enable the surgeon to view anatomical structures in detail and perform precise surgical interventions. The system 100 is typically utilized to perform minimally invasive surgeries, although this is not required. The system 100 requires smaller incisions compared to traditional open surgery, which results in reduced trauma, less blood loss, smaller scars, and faster recovery times for patients. The system 100 includes capabilities for remote manipulation, such that a surgeon located remove from the system 100 can still instruct the system on how to implement certain surgical procedures.

The system 100 include a primary controller 102 and one or more base controllers 104 in communication with the primary controller 102. Each of the one or more base controllers 104 may be configured to instruct the movements of multiple robotic arms 106, as shown in FIG. 1, wherein the example base controller 104 is in communication with three independent robotic arms 106. Each robotic arm 106 includes a tool receptacle 108 wherein a surgical instrument 110 may be installed. However, in most implementations, each robotic arm 106 includes one tool receptacle 108 and thus manages one surgical instrument 110. Each surgical instrument 110 is equipped with a tool drive adaptor 112 that enables mechanical and electronic coupling between the surgical instrument 110 and the tool receptacle 108, which thereby enables electronic coupling between the surgical instrument 110 and the controllers 102, 104.

The robotic arms 106 are located at a surgical robotic platform such as a table or bed. Each of the robotic arms 106 may be installed within a table-mounted system, but in other configurations, the robotic arms 106 may be mounted in a cart, ceiling or sidewall, or other suitable support surface. Generally, a user, such as a surgeon or other operator, may utilize a user console rendered by the primary controller 102 to remotely manipulate the robotic arms 106 and/or surgical instruments 110. The user console may be located in the same operating room as the system 100, located in an adjacent or nearby room, or teleoperated from a remote location in a different building, city, or country.

The robotic arms 106 are equipped with a tool receptacle 108 that is configured to receive a surgical instrument 110. The tool receptacle 108 comprises electronic communication pads to provide instructions to the surgical instrument 110 and the tool drive adaptor 112. The electronic communication pads further enable the tool receptacle 108 to receive data from the surgical instrument 110, which in some cases, may include real-time visualization data output by a camera affixed to the surgical instrument 110. This data is provided to the base controller 104 and/or the primary controller 102 for further processing. Additionally, the electronic communication established between the surgical instrument 110 and the tool receptacle 108 enables the primary controller 102 to identify the type of surgical instrument 110 that has been installed into the tool receptacle 108. The system 100 can read and write data to the electronic communication pads, including, for example, tool identification data, calibration data, tool life data, tool use data, tool sterilization data, and so forth.

The surgical instruments 110 may be installed and removed from the tool receptacles 108 such that diverse types of surgical instruments may be utilized by each of the robotic arms 106. A surgeon may select and request that certain surgical instruments 110 be installed in certain robotic arms 106 depending on the scope of needs of the surgical procedure to be performed. The surgical instruments 110 may be removed and replaced during a surgical operation as needed. Additionally, the surgical instruments 110 may be removed for reprocessing and sterilization procedures. The surgical instruments 110 might include, for example, one or more of an endoscope comprising a camera, a retractor, a cauterizer, a stapler, a cutter, a scalpel, and so forth. The endoscope comprising the camera may have any suitable configuration depending on the surgical operation to be performed. For example, the endoscope may specifically be any of a laparoscope, laryngoscope, colonoscope, bronchoscope, sigmoidoscope, and so forth.

The surgical instruments 110 are equipped with a tool drive adaptor 112 that enables the base controller 104 (by way of the robotic arm 106) to control movements of the surgical instrument 110. The base controller 104 controls movements of the robotic arms 106 and provides a communication channel between the primary controller 102 and the tool receptacle 108, which enables the tool receptacle 108 to actuate movement of the surgical instrument 110. The tool receptacle 108 provides rotational movement of the surgical instrument 110 and additionally provides axial movement of the surgical instrument 110 relative to the robotic arm 108. The robotic arm 106 is capable of moving within a spherical coordinate system. The tool drive adaptor 112 described herein is specifically implemented to control the rotational movements of the surgical instrument 110. This may be particularly useful when the surgical instrument includes a rigid scope that needs to be rotated clockwise or counterclockwise during the surgical operation.

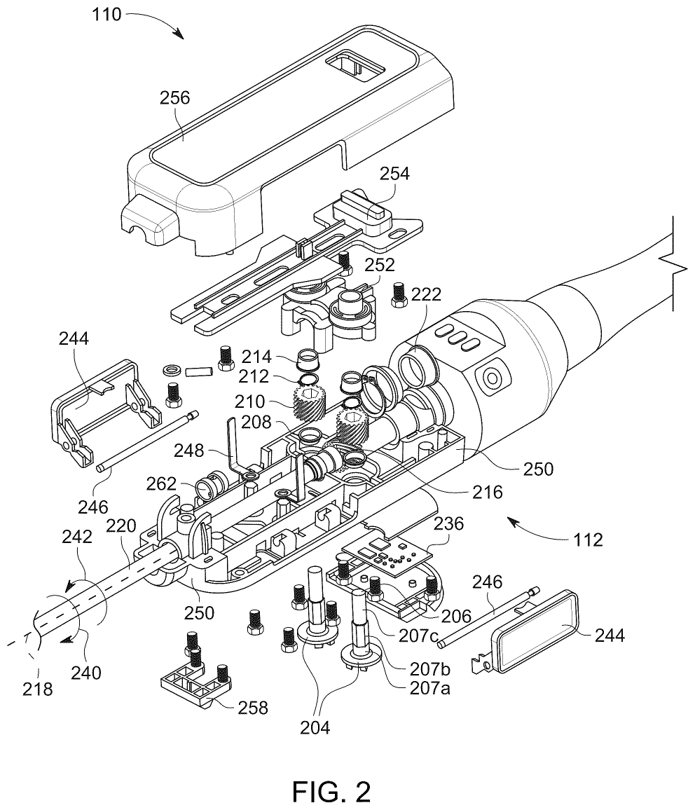

FIGS. 2-4 each illustrate exploded perspective views of a portion of a surgical instrument 110 comprising a tool drive adaptor 112. FIGS. 2, 3, and 4 provide varying views of the surgical instrument 110 with varying components exploded out to provide a comprehensive understanding of the tool drive adaptor 112. The tool drive adaptor is configured to interface with a tool receptacle 108 attached to a robotic arm 106. The tool drive adaptor 112 enables the system 100 to communicate with the surgical instrument 110 and control rotational movement of the surgical instrument 110.

The tool drive adaptor 112 includes two or more input pucks 204. When the tool drive adaptor 112 is successfully connected to the system 100 by way of the tool receptacle 108, the two or more input pucks 204 are coupled to corresponding motors on the robotic arm 106. The tool receptacle 108 attached to the robotic arm 106 includes two separate motors that are each configured to drive the rotational movement of an input puck 204. Specifically, the tool receptacle 108 includes a first motor that is mechanically coupled to a first input puck 204, and further includes a second motor that is mechanically coupled to a second input puck 204.

Each of the input pucks 204 is attached to a drive shaft 206 such that the drive shafts 206 are also coupled to the corresponding motors of the robotic arm 106. In some embodiments, the input puck 204 is a separate component that is attached to the drive shaft 206. In other embodiments, the input puck 204 and the drive shaft 206 for a single indivisible component. The input pucks 204 are coupled to motor drive shafts within the tool receptacle 108 such that the motors of the tool receptacle 108 can drive rotational movement of the drive shafts 206.

Each drive shaft 206 is configured to be disposed within a helical gear bearing 208, a helical gear 210, a clip 212, and an axle bearing 214. The helical gear 210 is keyed into the drive shaft 206 and is disposed between the helical gear bearing 208 and the axle bearing 214. The clip 212 locks the vertical gravel of the helical gear 210. The helical gear bearing 208 facilitates rotation of the drive shaft 206 and distributes the load to provide further stability. The drive shaft 206 is configured to form an interference fit with a corresponding internal geometry of the helical gear 210. This enables the interference fit between the drive shaft 206 and the helical gear 210 to cause rotation of the helical gear 210 when the drive shaft 206 is rotated by a corresponding motor of the tool receptacle 108 of the system 100.

The drive shaft 206 includes a proximal external geometry 207a (i.e., proximal to the motor of the robotic arm 106 of the system 100, which is not shown in FIG. 2), a middle external geometry 207b, and a distal external geometry 207c (i.e., distal to the motor of the robotic arm 106 of the system 100 relative to the proximal external geometry 207a). The proximal external geometry 207a is optimized for being disposed within the helical gear bearing 208. The middle external geometry 207b is optimized for forming an interference fit with an internal geometry of the helical gear 210. In some cases, rather than forming only an interference fit, the middle external geometry 207b is optimized to key the helical gear 210 to the drive shaft 206. The distal external geometry 207c is optimized for being disposed within the clip 212 and the axle bearing 214 and further to assist assembly of the clip 212 to lock the vertical travel of the helical gear 210.

The middle external geometry 207b corresponds with the internal surface, or internal geometry, of the bore of the helical gear 210. Thus, the middle external geometry 207b is keyed to the internal surface of the helical gear 210 to enable the drive shaft 206 to transfer rotational movement to the helical gear 210.

In the implementation illustrated in FIG. 2, the middle external geometry 207b of the drive shaft 206 comprises a hexagonal cross-sectional geometry. This portion of the drive shaft 206 may thus be referred to as a hex drive shaft. In this implementation, the helical gear 210 comprises a bore having a hexagonal cross-sectional geometry that corresponds with the hexagonal cross-sectional geometry of the middle external geometry 207b of the drive shaft 206. It should be understood that the middle external geometry 207b and the bore of the helical gear 210 may have alternative geometries that still cause the drive shaft 206 to rotate the helical gear 210 when the drive shaft 206 is rotated by the motor of the robotic arm 106 of the system 100.

The helical gear bearing 208 comprises an inner cross-sectional geometry that corresponds with the proximal external geometry 207a of the drive shaft 206. The helical gear bearing 208 aids in distributing the mechanical load and providing stability to the helical gear 210.

The helical gear 210 is mechanically coupled to the motor of the tool receptacle 108 of the system 100 by way of the input puck 204 and the drive shaft 206. The helical gear 210 comprises teeth cut at an angle, which results in a larger contact area between teeth. This enables the helical gear 210 to manage higher loads and transmit more torque compared with other gears of the same size. This is particularly beneficial when the tool drive adapter 202 is used to rotate a heavy device such as a surgical laparoscope. Additionally, including two or more helical gears 210 within the tool drive adaptor 112 enables the dual drive shafts 206 to rotate a heavy surgical instrument 110.

The helical gear 210 engages with a rotor gear 216 that is attached to the instrument shaft 220. The rotor gear 216 is oriented perpendicular relative to the helical gear 210 and the drive shaft 206. The rotor gear 216 is disposed substantially parallel to a longitudinal axis 218 of the instrument shaft 220. The helical gear 210 and the rotor gear 216 may be implemented with different gear types without departing from a scope of the disclosure. In alternative embodiments, the pair of gears 210, 216 may be implemented as a pair of perpendicular straight bevel gears, perpendicular spiral bevel gears, perpendicular miter gears, or a worm gear and worm wheel. The geometries of the gears 210, 216 may be selected based on the anticipated load for rotating the instrument shaft 220 clockwise 242 or counterclockwise 240.

The clip 212 is disposed between the helical gear 210 and the axle bearing 214. The clip 212 is configured to distribute the load, provide stability to the helical gear 210 and the axle bearing 214, and further to restrict the vertical movement of the helical gear 210. The axle bearing 214 supports and facilitates rotation of the drive shaft 206 while minimizing friction and wear. The axle bearing 214 aids in distributing the load evenly along a length of the drive shaft 206 to prevent excessive stress on specific points of the drive shaft 206, and to aid in smooth and stable operation of the pair of gears 210, 216.

When the motor of the robotic arm 106 of the system 100 causes the drive shaft 206 to rotate, the drive shaft 206 thereby causes the helical gear 210 to rotate, and then the helical gear 210 causes the rotor gear 216 to rotate. When the rotor gear 216 rotates, the instrument shaft 220 also rotates abouts its longitudinal axis 218. These components of the tool drive adaptor 112 thus enable the robotic arm 106 to finely control the rotational position of the instrument shaft 220 while the endoscopic tool is in use (based on instructions from one or more of the primary controller 102 or the base controller 104).

The rotation of the instrument shaft 220 is facilitated with an axle bearing 222 attached to the instrument shaft 220. The axle bearing 222 facilitates rotation of the instrument shaft 220 at a proximal end of the tool drive adaptor 112, and an axle bearing 262 facilitates rotation of the instrument shaft 220 at a distal end of the tool drive adaptor 112. The axle bearing 222 associated with the rotor gear 216 is similar to the axle bearings 210, 214 associated with the helical gears 210. The axle bearings 210, 214, 222, 262 each provide both axial and radial stability to their respective axle shafts. They aid in maintaining proper alignment and positioning of the axle, and thus prevent excessive axial or radial movement that could lead to vibration, misalignment, or premature wear.

The tool drive adaptor 112 additionally includes an equipment communication subsystem (ECSS) 236. The ECSS 236 facilitates electronic communication between the surgical instrument 110 and the base controller 104 or primary controller 102 (by way of the tool receptacle 108 and the robotic arm 106). When the surgical instrument 110 is installed into the tool receptacle 108, the ECSS 236 communicates with the robotic surgical system by way of the tool receptacle 108. Specifically, the ECSS 236 provide an identification of the endoscopic tool to the robotic surgical system, which may include an identification of what type of device it is (e.g., camera, cauterizer, stapler, cutter, retractor, and so forth) and may further include the device's serial number. Additionally, the ECSS 236 provides the configuration file for the endoscopic tool to a controller 102, 104 of the system 100 (by way of the electronic connection between the surgical instrument and the tool receptacle 108). A controller 102, 104 of the system 100 then reads the configuration file prior to driving the input pucks 204 to drive rotation of the instrument shaft 220.

The surgical instrument 110 includes a pair of latches 244, latch pins 246, and latch springs 248 for releasing the surgical instrument 110 from the tool receptacle 108 of the system 100 (not shown in FIG. 2). When a user wishes to install the surgical instrument 110 within the tool receptacle 108, the user will place the surgical instrument 110 into the tool receptacle 108 and ensure the surgical instrument 110 latches into place. In some implementations, the user will slide the surgical instrument 110 into the tool receptacle 108 and then allow the pair of latches 244 to engage.

When the user wishes to remove the surgical instrument 110 from the tool receptacle 108, the user will simultaneously pinch the latches 244 on either side of the surgical instrument 110 to depress the latches 244 inward toward the instrument shaft 220. The user may then lift or slide the surgical instrument 110 out of the tool receptacle 108. The surgical instrument 110 may then be utilized in a different robotic arm 106, reprocessed and/or sterilized, used directly by a user as a handheld device, and so forth.

The surgical instrument 110 includes a chassis 250 for the tool drive adaptor 112. The chassis 250 also serves as a lower housing for protecting components of the tool drive adaptor and gear train. This lower housing/chassis 250 corresponds with an upper housing 256 to fully enclose the tool drive adaptor 220. The surgical instrument 110 includes a release slide 254 that aids in attaching and detaching the upper housing from the lower housing. The release slide 254 and the upper housing 256 enable a user to remove the upper housing 256 (typically manufactured of a plastic material) from the surgical instrument 110 prior to sterilizing the remaining components of the surgical instrument 110.

The surgical instrument 110 includes a gear box cover 252 to protect components of the gear box, including the axle bearing 214, clip 212, helical gear 210, gear washer 208, and drive shaft 206. The gear box cover 252 further protects the interface between the helical gear 210 and the rotor gear 216.

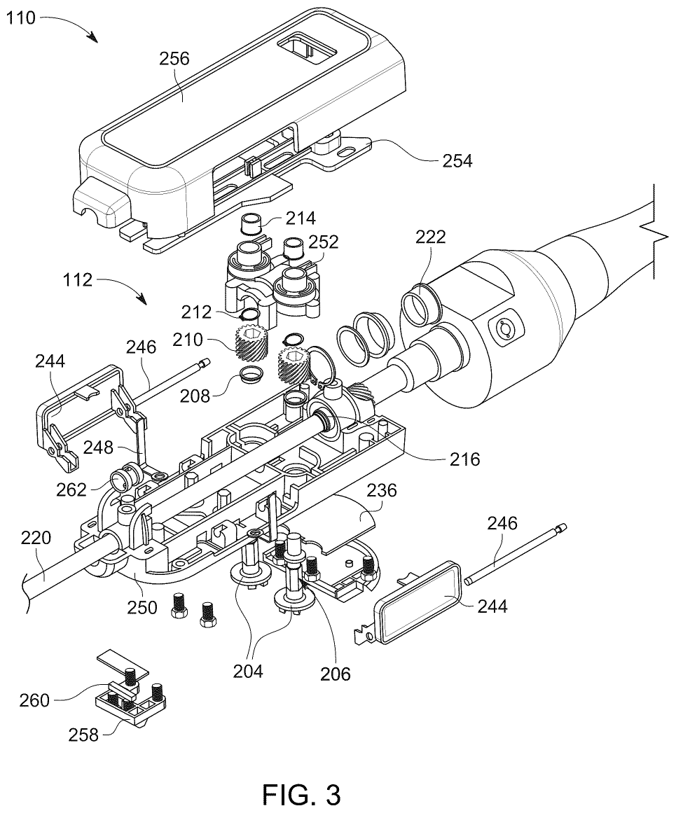

FIG. 3 is an exploded perspective view of the surgical instrument 110 including the tool drive adaptor 112 and the instrument shaft 220. The exploded perspective view illustrated in FIG. 2 is slightly modified relative to the exploded perspective view illustrated in FIG. 2 to provide an alternate view of certain components.

As shown in FIG. 3, the surgical instrument 110 includes a magnet assembly disposed at a distal end of the chassis 250 for the tool drive adaptor 112 (distal relative to the robotic arm 106). The magnet assembly includes a magnet 260 disposed within a magnet housing subassembly 258. The magnet assembly is utilized to determine whether the surgical instrument 110 is properly attached to the tool receptacle 108 of the system 100 (not shown in FIG. 2). The tool receptacle 108 includes a Hall effect sensor that detects a presence and position of the magnet 260. When the magnet 260 is sufficiently near the Hall effect sensor of the tool receptacle 108, the surgical instrument 110 is classified as being fully installed within the tool receptacle 108.

The Hall effect sensor (not shown) is an electronic device designed to detect the Hall effect and convert its findings into electronic data. If the magnet 260 is placed perpendicular to a conductor with a steady flow of current (wherein the conductor is a component of the Hall effect sensor within the tool receptacle 108), the electrons flowing within the conductor are pulled to one side, thus creating a potential difference in charge (i.e., voltage). The Hall effect, then, is indicative of the presence and magnitude of the magnet 260 near the conductor. The Hall effect sensor of the tool receptacle 108 may serve as a Hall effect switch or latch. The Hall effect sensor may provide electronic data to a controller 102, 104 of the robotic surgical system 100 to indicate that the surgical instrument 110 is successfully installed and the robotic surgical system may now engage the tool drive adaptor 112 and utilize the surgical instrument 110.

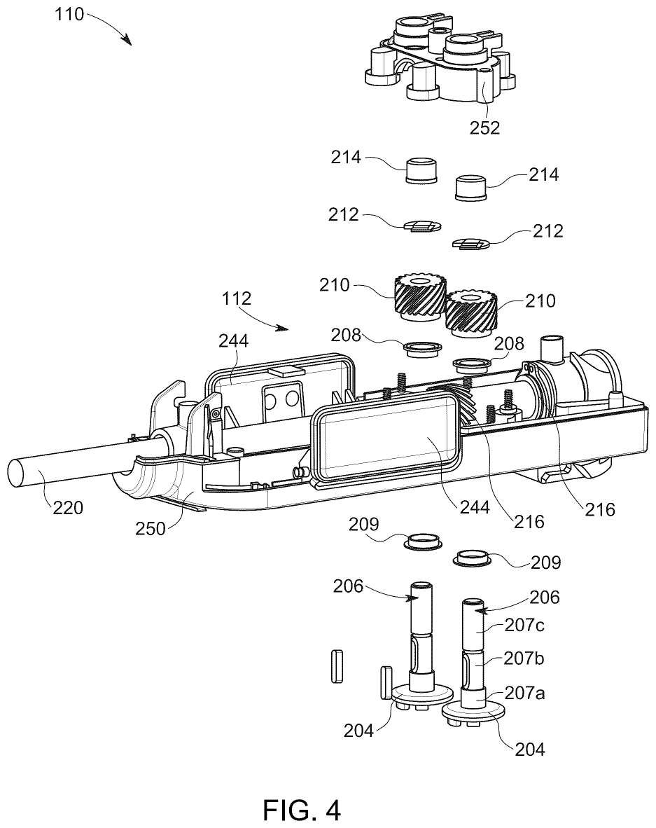

FIG. 4 illustrates an exploded perspective view of the surgical instrument 110 comprising the tool drive adaptor 112. FIG. 4 specifically illustrates how various components of the tool drive adaptor 112 are stacked vertically from the input pucks 204 up through the axle bearings 214.

As shown specifically in FIG. 4, the driving force for rotating the instrument shaft 220 includes a plurality of vertically stacked components. These vertically stacked components begin at the bottom with the input pucks 204, which are configured to interface with corresponding motors on the tool receptacle 108. The input pucks 204 are each attached to a corresponding drive shaft 206. The drive shafts 206 are configured to be disposed through holes cut into the lower housing of the tool drive adaptor 112. The lengths of the drive shafts 206 may be optimized such that the drive shafts 206 extend vertically through all components of the tool drive adaptor 112 and can be seen at a top portion of the gear box cover 252.

Continuing through the vertically-stacked components of the tool drive adaptor 112, there are numerous components that are slid down on to the drive shafts 206. These include an underside helical gear bearing 209, which is referred to as the “underside” helical gear bearing because it is located underneath the lower housing of the tool drive adaptor 112. The underside helical gear bearing 209 corresponds with the helical gear bearing 208, which is oriented in the opposite direction and is located above the lower housing of the tool drive adaptor 112. The corresponding pair of helical gear bearings 209, 208 are followed by the helical gears 210. The helical gears 210 are then followed by the clips 212 and axle bearings 214. Finally, the gear box cover 252 serves as the top-most portion of the tool drive adaptor 112 and is configured to cover and protect several of the vertically-stacked components.

The bottom of the tool drive adaptor 112 includes the two or more input pucks 204 and the corresponding drive shafts 206 attached thereto. FIG. 4 specifically illustrates the optimized geometry of the drive shafts 206, including the proximal external geometry 207a, the middle external geometry 206b, and the distal external geometry 207c. In various implementations described herein, any of the proximal, middle, or distal external geometries 207a, 207b, 207c of the drive shaft 206 may have a hexagonal or elliptical (as described herein, “elliptical” includes circular) cross-sectional geometry. As shown in FIG. 4, each of the external geometries 207a, 207b, 207c has an elliptical/circular cross-sectional geometry. However, in alternate implementations, any one or more of the external geometries 207a, 207b, 207c have may a hexagonal cross-sectional geometry.

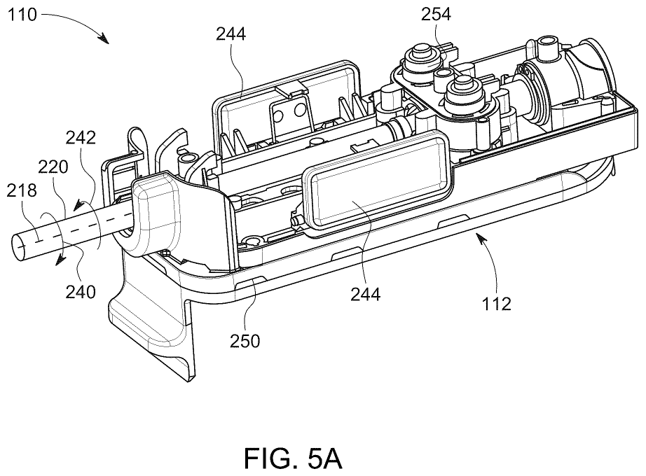

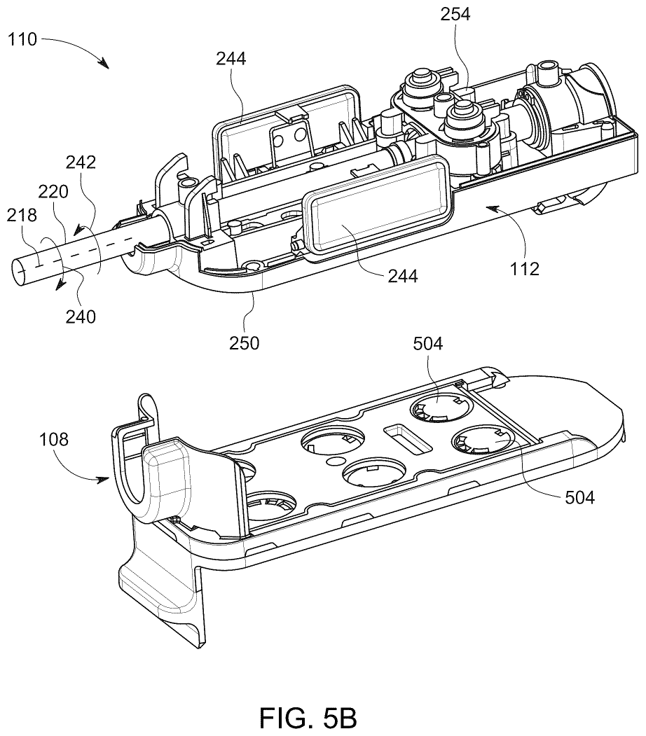

FIGS. 5A and 5B illustrate perspective views of the surgical instrument 110 comprising the tool drive adaptor 112. FIG. 5A specifically illustrates the surgical instrument 110 and tool drive adaptor 112 alone, and FIG. 5B illustrates the surgical instrument 110 and tool drive adaptor 112 hovering over a corresponding tool receptacle 108 of the system 100.

FIGS. 5A and 5B illustrate wherein the components of the tool drive adaptor 112, including the input puck 204, drive shaft, 206, helical gear bearing 208, helical gear 210, clip 212, and axle bearing 214 are vertically stacked to enable rotational movement of the instrument shaft 220.

As shown in FIG. 5B, the surgical instrument 110 and tool drive adaptor 112 are configured to be locked into a corresponding tool receptacle 108. The tool receptacle 108 includes coupling components 504 that are configured to interface with and couple to the input pucks 204 of the tool drive adaptor 112. The coupling components 504 include a means to lock into the input pucks 204 and thus drive rotation of the input pucks 204. The coupling components 504 additionally include a means for electronic communication with the surgical instrument 110. This electronic communication enables bidirectional communication between the surgical instrument 110 and the primary controller 102 and/or base controller 104.

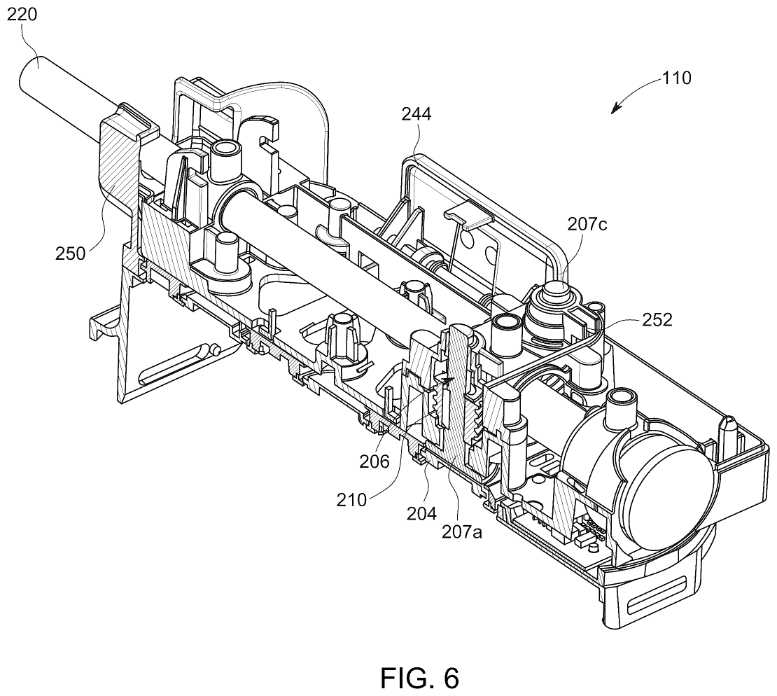

FIG. 6 illustrates a perspective cross-sectional view of the surgical instrument 110 comprising the tool drive adaptor 112. FIG. 6 specifically illustrates wherein approximately one-third of the tool drive adaptor 112 is sliced off along a longitudinal axis of the instrument shaft 220. This illustrates the internal components of the tool drive adaptor 112, including the helical gear 210 disposed around the drive shaft 206.

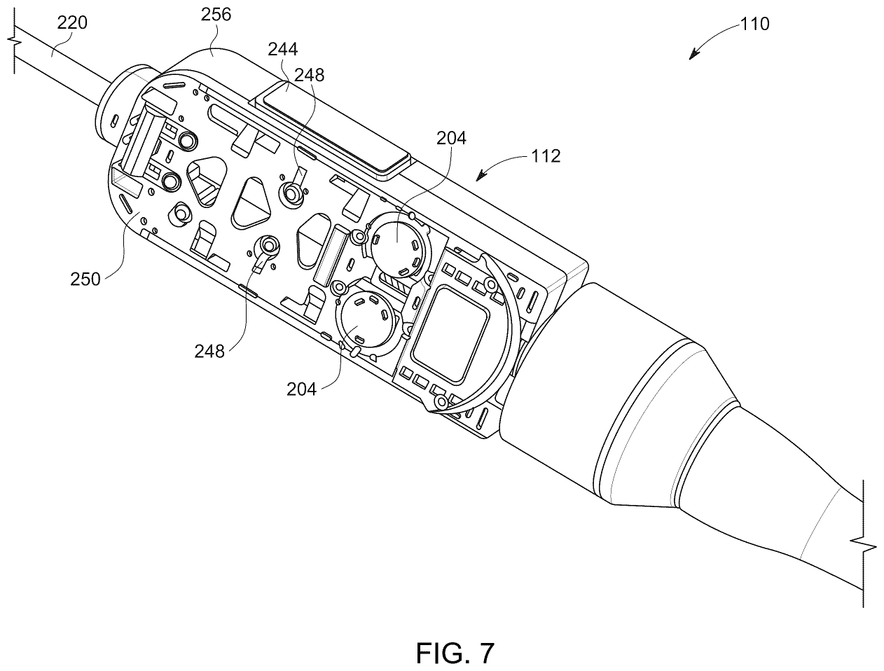

FIG. 7 is a perspective view of an underside of a portion of the surgical instrument 110, and specifically illustrates an underside of the tool drive adaptor 112. FIG. 7 illustrates wherein the upper housing 256 is installed on to the chassis 250 such that the components of the tool drive adaptor 112 are enclosed.

One of the two latches 244 is visible in FIG. 7 wherein it is installed into a sidewall formed by the upper housing 256 and the chassis 250. As shown in FIG. 7, the chassis 250 includes holes disposed through a base of the chassis 250 to allow access to the latch springs 248. The latch springs 248 are configured to releasably affix to corresponding latches on the tool receptacle 108. When the latch 244 is depressed, the latch spring 248 release from the corresponding latches on the tool receptacle 108, and the surgical instrument 110 may then be lifted out of the tool receptacle 108.

The latch of the tool drive adaptor 112 includes a leg formation configured to extend beyond a bottom face of the lower housing when pinched or compressed. This facilitates the release of the surgical instrument 110 from the tool receptacle 108. The latch springs 248 facilitate the release of the latch back to an initial position after the surgical instrument 110 is detached from the tool receptacle 108. The latch springs 248 may be implemented with varying design parameters depending on the implementation. Specifically, the latch springs 248 may include one or more of a leaf spring, torsion spring, compression spring, extension spring, or another suitable spring type.

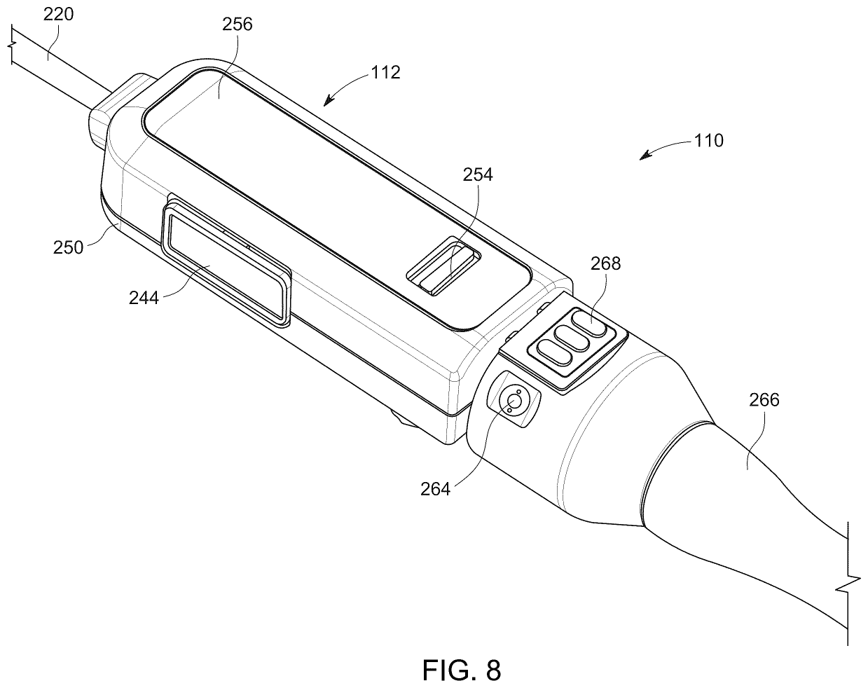

FIG. 8 is a perspective view of a top side of a portion of the surgical instrument 110, and specifically illustrates the top side of the tool drive adaptor 112. FIG. 8 illustrates wherein the upper housing 256 is installed on to the chassis 250 such that the components of the tool drive adaptor 112 are enclosed.

In FIG. 8, a portion of the release slide 254 is shown pressed through a hole cut through a top side of the upper housing 256. A use may pull the release slide down (i.e., in a direction toward the instrument shaft 220) to release the upper housing 256 from the chassis 250. This enables quick access to the tool drive adaptor 112. Additionally, this enables a user to remove the upper housing 256 prior to reprocessing or sterilizing the remaining components of the surgical instrument 110.

The surgical instrument 110 may include its own indication light 264 that indicates whether the surgical instrument 110 is properly plugged in and connected to power. Additionally, the surgical instrument 110 may include one or more actuator buttons 268 for manipulating the surgical instrument 110. In some cases, when the surgical instrument 110 is a visualization device, the actuator buttons 268 may be utilized to select different visualization outputs as discussed further herein. For example, a user may request that one or more of color, fluorescence, or spectral imaging be displayed on a screen, which overlay images be displayed on a screen, and/or that dimensional information be provided.

The surgical instrument 110 may be used in connection with the robotic surgical system 100 or may be utilized as a handheld device. When the surgical instrument 110 is used as a handheld device, the user may manually rotate the instrument shaft 220 by rotating the body of the surgical instrument 110. In these cases, the tool drive adaptor 112 will be “locked” such that the tool drive adaptor will not impact the rotational movement of the instrument shaft 220.

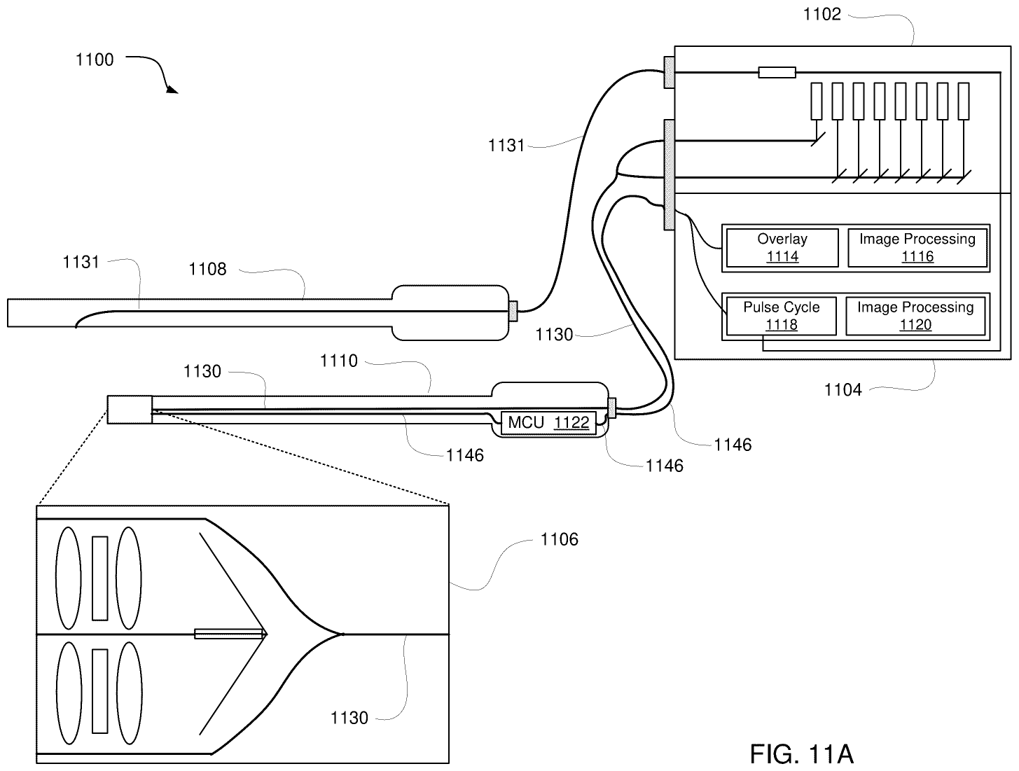

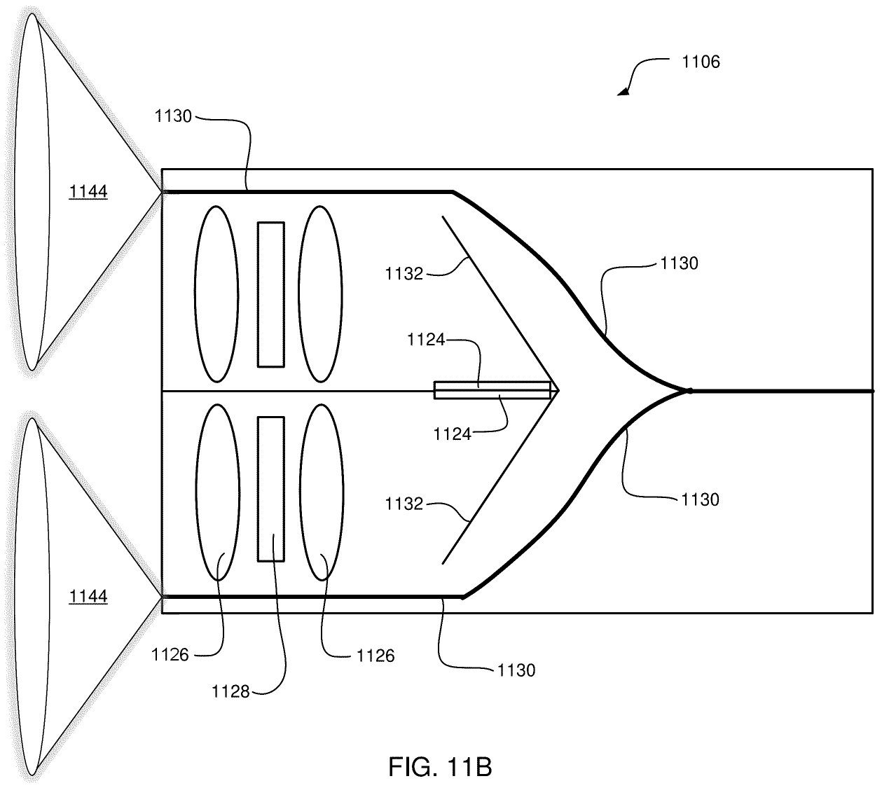

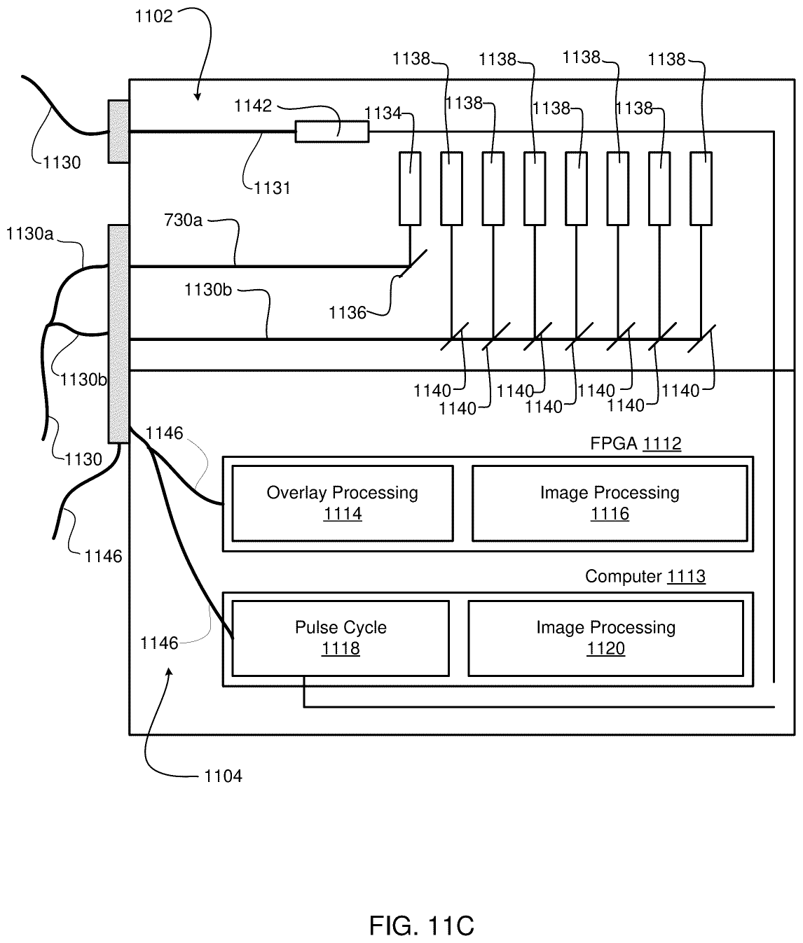

The surgical instrument 110 includes a cable 266 that enables bidirectional electronic communication between components of the surgical instrument and a controller 102, 104 of the robotic surgical system 100. The cable 266 may additionally enable bidirectional electronic communication between components of the surgical instrument and a visualization controller as discussed further herein (see, e.g., FIGS. 11A-11C).

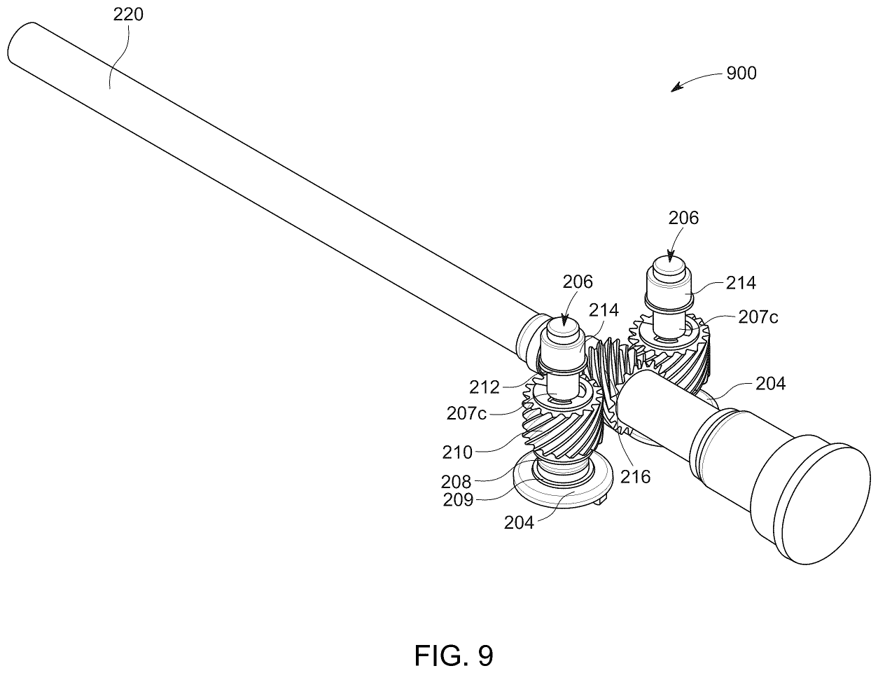

FIG. 9 is a perspective view of a gear assembly 900 of the tool drive adaptor 112. The gear assembly 900 includes the drive shafts 206, the plurality of components disposed around the drive shafts 206, and the input pucks 204 that are configured to receive rotational movement from a motor disposed within a corresponding tool receptacle 108. The gear assembly 900 specifically includes the underside helical gear bearing 209, the helical gear bearing 208, the helical gear 210, the clip 212, and the axle bearing 212 stacked vertically around the drive shafts 206. The gear assembly 900 additionally includes the rotor gear 216 disposed around the instrument shaft 220. As shown in FIG. 9, the pair of helical gears 210 are configured to interface with the rotor gear 216. The rotation of the pair of helical gears 210 causes rotation of the rotor gear 216 and thereby causes rotation of the instrument shaft 220.

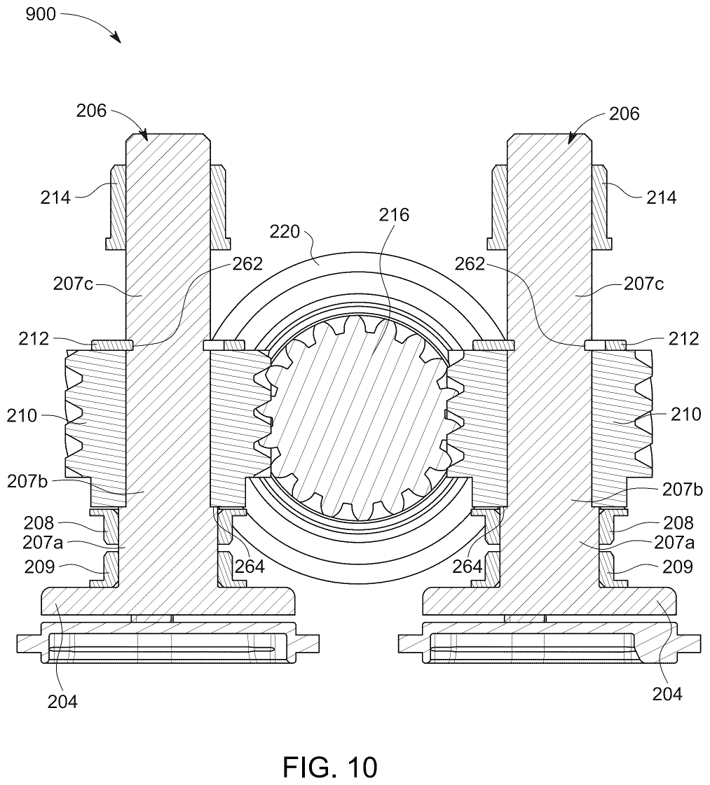

FIG. 10 is a cross-sectional straight-on view of the gear assembly 900 of the tool drive adaptor. FIG. 10 specifically illustrates a straight-on view staring down the longitudinal axis of the instrument shaft 220. As shown in FIG. 10, each of the two helical gears 210 engages with the rotor gear 216 and thereby causes rotation of the rotor gear 216.

FIGS. 11A-11C illustrate schematic diagrams of a system 1100 for endoscopic visualization. The system 1100 provides visualization for the system 100 for robotic surgery or robot-assisted surgery that is described herein. The visualization data captured by the system 110 may be utilized by a surgeon or algorithm when generating instructions to be executed by the primary controller 102 and/or base controller 104 of the system 100.

The system 1100 includes an emitter 1102, a controller 1104, and an optical visualization system 1106. The system 1100 includes one or more tools 1108, which may include endoscopic tools such as forceps, brushes, scissors, cutters, burs, staplers, ligation devices, tissue staplers, suturing systems, and so forth. The system 1100 includes one or more endoscopes 1110 such as arthroscopes, bronchoscopes, colonoscopes, colposcopes, cystoscopes, esophagoscope, gastroscopes, laparoscopes, laryngoscopes, neuroendoscopes, proctoscopes, sigmoidoscopes, thoracoscopes, and so forth. Each of the one or more tools 1108 and the one or more endoscopes 1110 is a surgical instrument 110 as discussed herein and will be disposed into a tool receptacle 108 of the system 100.

The optical visualization system 1106 may be disposed at a distal end of a lumen of an endoscope 1110. Alternatively, one or more components of the optical visualization system 1106 may be disposed at a proximal end of the lumen of the endoscope 1110 or in another region of the endoscope 1110. The optical visualization system 1106 may include one or more image sensors 1124 that each include a pixel array. The optical visualization system 1106 may include one or more lenses 1126 and filters 1128 and may further include one or more prisms 1132 for reflecting EMR on to the pixel array 1125 of the one or more image sensors 1124. The system 1100 may include a waveguide 1130 configured to transmit EMR from the emitter 1102 to a distal end of the endoscope 1110 to illuminate a light deficient environment for visualization, such as within a surgical scene. The system 1100 may further include a waveguide 1131 configured to transmit EMR from the emitter 1102 to a termination point on the tool 1108, which may specifically be actuated for laser mapping imaging and tool tracking as described herein.

The optical visualization system 1106 may specifically include two lenses 1126 dedicated to each image sensor 1124 to focus EMR on to a rotated image sensor 1124 and enable a depth view. The filter 1128 may include a notch filter configured to block unwanted reflected EMR. In a particular use-case, the unwanted reflected EMR may include a fluorescence excitation wavelength that was pulsed by the emitter 1102, wherein the system 1100 wishes to only detect a fluorescence relaxation wavelength emitted by a fluorescent reagent or tissue.

The image sensor 1124 includes one or more image sensors, and the example implementation illustrated in FIGS. 11A-11B illustrates an optical visualization system 1106 comprising two image sensors 1124. The image sensor 1124 may include a CMOS image sensor and may specifically include a high-resolution image sensor configured to read out data according to a rolling readout scheme. The image sensors 1124 may include a plurality of different image sensors that are tuned to collect different wavebands of EMR with varying efficiencies. In an implementation, the image sensors 1124 include separate image sensors that are optimized for color imaging, fluorescence imaging, multispectral imaging, and/or topographical mapping.

The emitter 1102 includes one or more EMR sources, which may include, for example, lasers, laser bundles, light emitting diodes (LEDs), electric discharge sources, incandescence sources, electroluminescence sources, and so forth. In some implementations, the emitter 1102 includes at least one white EMR source 1134 (may be referred to herein as a white light source). The emitter 1102 may additionally include one or more EMR sources 1138 that are tuned to emit a certain waveband of EMR. The EMR sources 1138 may specifically be tuned to emit a waveband of EMR that is selected for multispectral or fluorescence visualization. The emitter 1102 may additionally include one or more mapping sources 1142 that are configured to emit EMR in a mapping pattern such as a grid array or dot array selected for capturing data for topographical mapping or anatomical measurement.

The one or more white EMR sources 1134 emit EMR into a dichroic mirror 1136 that feeds the white EMR into a waveguide 1130, which may specifically include a fiber optic cable or other means for carrying EMR to the endoscope. The white EMR source 1134 may specifically feed into a first waveguide 1130a dedicated to white EMR. The EMR sources 1138 emit EMR into independent dichroic mirrors 1140 that each feed EMR into the waveguide 1130 and may specifically feed into a second waveguide 1130b. The first waveguide 1130a and the second waveguide 1130b later merge into a waveguide 1130 that transmits EMR to a distal end of the endoscope 1110 to illuminate a scene with an emission of EMR 1144.

The one or more EMR sources 1138 that are tuned to emit a waveband of EMR may specifically be tuned to emit EMR that is selected for multispectral or fluorescence visualization. In some cases, the EMR sources 1138 are finely tuned to emit a central wavelength of EMR with a tolerance threshold not exceeding ±5 nm, ±4 nm, ±3 nm, ±2 nm, or ±1 nm. The EMR sources 1138 may include lasers or laser bundles that are separately cycled on and off by the emitter 1102 to pulse the emission of EMR 1144 and illuminate a scene with a finely tuned waveband of EMR.

The one or more mapping sources 1142 are configured to pulse EMR in a mapping pattern, which may include a dot array, grid array, vertical hashing, horizontal hashing, pin grid array, and so forth. The mapping pattern is selected for laser mapping imaging to determine one or more of a three-dimensional topographical map of a scene, a distance between two or more objects within a scene, a dimension of an object within a scene, a location of a tool 1108 within the scene, and so forth. The EMR pulsed by the mapping source 1142 is diffracted to spread the energy waves according to the desired mapping pattern. The mapping source 1142 may specifically include a device that splits the EMR beam with quantum-dot-array diffraction grafting. The mapping source 1142 may be configured to emit low mode laser light.

The controller 1104 (may be referred to herein as a camera control unit or CCU) may include a field programmable gate array (FGPA) 1112 and a computer 1113. The FGPA 1112 may be configured to perform overlay processing 1114 and image processing 1116. The computer 1113 may be configured to generate a pulse cycle 1118 for the emitter 1102 and to perform further image processing 1120. The FGPA 1112 receives data from the image sensor 1124 and may combine data from two or more data frames by way of overlay processing 1114 to output an overlay image frame. The computer 1113 may provide data to the emitter 1102 and the image sensor 1124. Specifically, the computer 1113 may calculate and adjust a variable pulse cycle to be emitted by the emitter 1102 in real-time based on user input. Additionally, the computer 1113 may receive data frames from the image sensor 1124 and perform further image processing 1120 on those data frames.

The controller 1104 may be in communication with a network, such as the Internet, and automatically upload data to the network for remote storage. The MCU 1122 and image sensors 1124 may be exchanged, updated, and continue to communicate with an established controller 1104. In some cases, the controller 1104 is “out of date” with respect to the MCU 1122 but will still successfully communicate with the MCU 1122. This may increase the data security for a hospital or other healthcare facility because the existing controller 1104 may be configured to undergo extensive security protocols to protect patient data.

The controller 1104 may communicate with a microcontroller unit (MCU) 1122 disposed within a handpiece of the endoscope and/or the image sensor 1124 by way of a data transmission pipeline 1146. The data transmission pipeline 1146 may include a data connection port disposed within a housing of the emitter 1102 or the controller 1104 that enables a corresponding data cable to carry data to the endoscope 1110. In another embodiment, the controller 1104 wirelessly communicates with the MCU 1122 and/or the image sensor 1124 to provide instructions for upcoming data frames. One frame period includes a blanking period and a readout period. Generally, the pixel array 1125 accumulates EMR during the blanking period and reads out pixel data during the readout period. It will be understood that a blanking period corresponds to a time between a readout of a last row of active pixels in the pixel array of the image sensor and a beginning of a next subsequent readout of active pixels in the pixel array. Additionally, the readout period corresponds to a duration of time when active pixels in the pixel array are being read. Further, the controller 1104 may write correct registers to the image sensor 1124 to adjust the duration of one or more of the blanking period or the readout period for each frame period on a frame-by-frame basis within the sensor cycle as needed.

The controller 1104 may reprogram the image sensor 1124 for each data frame to set a required blanking period duration and/or readout period duration for a subsequent frame period. In some cases, the controller 1104 reprograms the image sensor 1124 by first sending information to the MCU 1122, and then the MCU 1122 communicates directly with the image sensor 1124 to rewrite registers on the image sensor 1124 for an upcoming data frame.

The MCU 1122 may be disposed within a handpiece portion of the endoscope 1110 and communicate with electronic circuitry (such as the image sensor 1124) disposed within a distal end of a lumen of the endoscope 1110. The MCU 1122 receives instructions from the controller 1104, including an indication of the pulse cycle 1118 provided to the emitter 1102 and the corresponding sensor cycle timing for the image sensor 1124. The MCU 1122 executes a common Application Program Interface (API). The controller 1104 communicates with the MCU 1122, and the MCU 1122 executes a translation function that translates instructions received from the controller 1104 into the correct format for each type of image sensor 1124. In some cases, the system 1100 may include multiple different image sensors that each operate according to a different “language” or formatting, and the MCU 1122 is configured to translate instructions from the controller 1104 into each of the appropriate data formatting languages. The common API on the MCU 1122 passes information by the scene, including, for example parameters pertaining to gain, exposure, white balance, setpoint, and so forth. The MCU 1122 runs a feedback algorithm to the controller 1104 for any number of parameters depending on the type of visualization.

The MCU 1122 stores operational data and images captured by the image sensors 1124. In some cases, the MCU 1122 does not need to continuously push data up the data chain to the controller 1104. The data may be set once on the microcontroller 1122, and then only critical information may be pushed through a feedback loop to the controller 1104. The MCU 1122 may be set up in multiple modes, including a primary mode (may be referred to as a “master” mode when referring to a master/detail communication protocol). The MCU 1122 ensures that all downstream components (i.e., distal components including the image sensors 1124, which may be referred to as “detail components” in the master/detail communication protocol) are apprised of the configurations for upcoming data frames. The upcoming configurations may include, for example, gain, exposure duration, readout duration, pixel binning configuration, and so forth.

The MCU 1122 includes internal logic for executing triggers to coordinate different devices, including, for example multiple image sensors 1124. The MCU 1122 provides instructions for upcoming frames and executes triggers to ensure that each image sensor 1124 begins to capture data the same time. In some cases, the image sensors 1124 may automatically advance to a subsequent data frame without receiving a unique trigger from the MCU 1122.

In some cases, the endoscope 1110 includes two or more image sensors 1124 that detect EMR and output data frames simultaneously. The simultaneous data frames may be used to output a three-dimensional image and/or output imagery with increased definition and dynamic range. The pixel array of the image sensor 1124 may include active pixels and optical black (“OB”) or optically blind pixels. The optical black pixels may be read during a blanking period of the pixel array when the pixel array is “reset” or calibrated. After the optical black pixels have been read, the active pixels are read during a readout period of the pixel array. The active pixels accumulate EMR that is pulsed by the emitter 1102 during the blanking period of the image sensor 1124. The pixel array 1125 may include monochromatic or “color agnostic” pixels that do not comprise any filter for selectively receiving certain wavebands of EMR. The pixel array may include a color filter array (CFA), such as a Bayer pattern CFA, that selectively allows certain wavebands of EMR to pass through the filters and be accumulated by the pixel array.

The image sensor 1124 is instructed by a combination of the MCU 1122 and the controller 1104 working in a coordinated effort. Ultimately, the MCU 1122 provides the image sensor 1124 with instructions on how to capture the upcoming data frame. These instructions include, for example, an indication of the gain, exposure, white balance, exposure duration, readout duration, pixel binning configuration, and so forth for the upcoming data frame. When the image sensor 1124 is reading out data for a current data frame, the MCU 1122 is rewriting the correct registers for the next data frame. The MCU 1122 and the image sensor 1124 operate in a back-and-forth data flow, wherein the image sensor 1124 provides data to the MCU 1122 and the MCU 1122 rewrites correct registers to the image sensor 1124 for each upcoming data frame. The MCU 1122 and the image sensor 1124 may operate according to a “ping pong buffer” in some configurations.

The image sensor 1124, MCU 1122, and controller 1104 engage in a feedback loop to continuously adjust and optimize configurations for upcoming data frames based on output data. The MCU 1122 continually rewrites correct registers to the image sensor 1124 depending on the type of upcoming data frame (i.e., color data frame, multispectral data frame, fluorescence data frame, topographical mapping data frame, and so forth), configurations for previously output data frames, and user input. In an example implementation, the image sensor 1124 outputs a multispectral data frame in response to the emitter 1102 pulsing a multispectral waveband of EMR. The MCU 1122 and/or controller 1104 determines that the multispectral data frame is underexposed and cannot successfully be analyzed by a corresponding machine learning algorithm. The MCU 1122 and/or controller 1104 than adjusts configurations for upcoming multispectral data frames to ensure that future multispectral data frames are properly exposed. The MCU 1122 and/or controller 1104 may indicate that the gain, exposure duration, pixel binning configuration, and so forth, must be adjusted for future multispectral data frames to ensure proper exposure. All image sensor 1124 configurations may be adjusted in real-time based on previously output data processed through the feedback loop, and further based on user input.

The waveguides 1130, 1131 include one or more optical fibers. The optical fibers may be made of a low-cost material, such as plastic to allow for disposal of one or more of the waveguides 1130, 1131. In some implementations, one or more of the waveguides 1130, 1131 include a single glass fiber. In some implementations, one or more of the waveguides 1130, 1131 include a plurality of glass fibers.

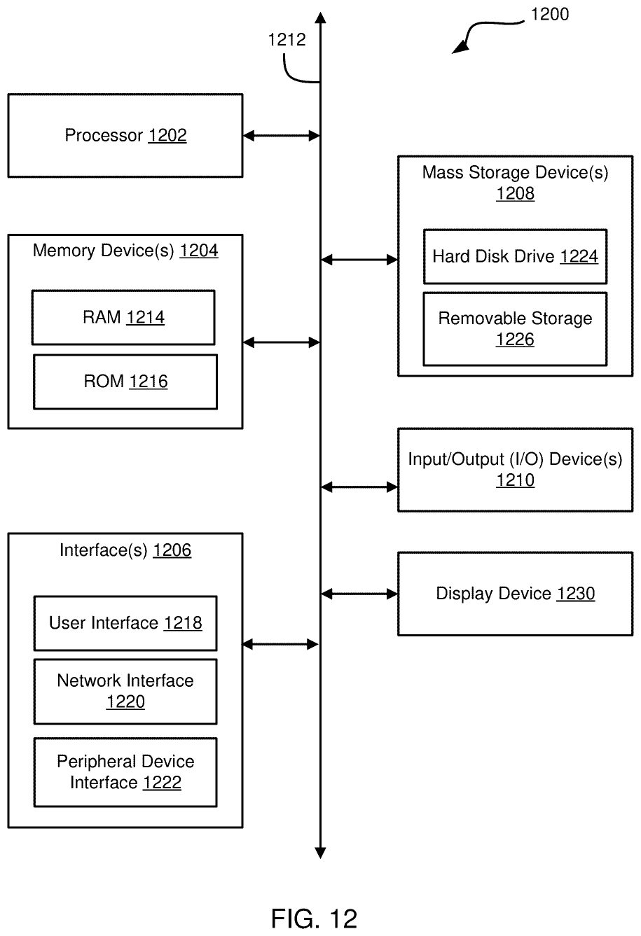

FIG. 12 illustrates a schematic block diagram of an example computing device 1200. The computing device 1200 may be used to perform various procedures, such as those discussed herein. The computing device 1200 can perform various monitoring functions as discussed herein, and can execute one or more application programs, such as the application programs or functionality described herein. The computing device 1200 can be any of a wide variety of computing devices, such as a desktop computer, in-dash computer, vehicle control system, a notebook computer, a server computer, a handheld computer, tablet computer and the like.

The computing device 1200 includes one or more processor(s) 1204, one or more memory device(s) 1204, one or more interface(s) 1206, one or more mass storage device(s) 1208, one or more Input/output (I/O) device(s) 1210, and a display device 1230 all of which are coupled to a bus 1212. Processor(s) 1204 include one or more processors or controllers that execute instructions stored in memory device(s) 1204 and/or mass storage device(s) 1208. Processor(s) 1204 may also include several types of computer-readable media, such as cache memory.

Memory device(s) 1204 include various computer-readable media, such as volatile memory (e.g., random access memory (RAM) 1214) and/or nonvolatile memory (e.g., read-only memory (ROM) 1216). Memory device(s) 1204 may also include rewritable ROM, such as Flash memory.

Mass storage device(s) 1208 include various computer readable media, such as magnetic tapes, magnetic disks, optical disks, solid-state memory (e.g., Flash memory), and so forth. As shown in FIG. 12, a particular mass storage device 1208 is a hard disk drive 1224. Various drives may also be included in mass storage device(s) 1208 to enable reading from and/or writing to the various computer readable media. Mass storage device(s) 1208 include removable media 1226 and/or non-removable media.

I/O device(s) 1210 include various devices that allow data and/or other information to be input to or retrieved from computing device 1200. Example I/O device(s) 1210 include cursor control devices, keyboards, keypads, microphones, monitors or other display devices, speakers, printers, network interface cards, modems, and the like.

Display device 1230 includes any type of device capable of displaying information to one or more users of computing device 1200. Examples of display device 1230 include a monitor, display terminal, video projection device, and the like.

Interface(s) 1206 include various interfaces that allow computing device 1200 to interact with other systems, devices, or computing environments. Example interface(s) 1206 may include any number of different network interfaces 1220, such as interfaces to local area networks (LANs), wide area networks (WANs), wireless networks, and the Internet. Other interface(s) include user interface 1218 and peripheral device interface 1222. The interface(s) 1206 may also include one or more user interface elements 1218. The interface(s) 1206 may also include one or more peripheral interfaces such as interfaces for printers, pointing devices (mice, track pad, or any suitable user interface now known to those of ordinary skill in the field, or later discovered), keyboards, and the like.

Bus 1212 allows processor(s) 1204, memory device(s) 1204, interface(s) 1206, mass storage device(s) 1208, and I/O device(s) 1210 to communicate with one another, as well as other devices or components coupled to bus 1212. Bus 1212 represents one or more of several types of bus structures, such as a system bus, PCI bus, IEEE bus, USB bus, and so forth.

For purposes of illustration, programs and other executable program components are shown herein as discrete blocks, such as block 302 for example, although it is understood that such programs and components may reside at various times in different storage components of computing device 1200 and are executed by processor(s) 1202. Alternatively, the systems and procedures described herein, including programs or other executable program components, can be implemented in hardware, or a combination of hardware, software, and/or firmware. For example, one or more application specific integrated circuits (ASICs) can be programmed to conduct one or more of the systems and procedures described herein.

### EXAMPLES

The following examples pertain to features of further embodiments:

Example 1 is a device for controlling rotational movement of a surgical instrument. The device includes a rotor gear attached to a shaft of the surgical instrument; a first helical gear rotatably coupled to the rotor gear, wherein the first helical gear comprises a first bore; a first drive shaft disposed through the first bore of the first helical gear; a second helical gear rotatably coupled to the rotor gear, wherein the second helical gear comprises a second bore; and a second drive shaft disposed through the second bore of the second helical gear.

Example 2 is a device as in Example 1, wherein the device is a tool drive adaptor that is mechanically coupled to a tool receptacle of a robotic surgical system to enable the robotic surgical system to control the rotational movement of the surgical instrument.

Example 3 is a device as in any of Examples 1-2, further comprising: a first input puck attached to the first drive shaft; and a second input puck attached to the second drive shaft; wherein a first motor of the tool receptacle is mechanically coupled to the first input puck to drive rotation of the first drive shaft; and wherein a second motor of the tool receptacle is mechanically coupled to the second input puck to drive rotation of the second drive shaft.

Example 4 is a device as in any of Examples 1-3, wherein a controller of the robotic surgical system provides instructions to a robotic arm of the robotic surgical system to actuate one or more of the first motor or the second motor of the tool receptacle.

Example 5 is a device as in any of Examples 1-4, wherein the controller of the robotic surgical system provides the instructions to actuate the one or more of the first motor or the second motor in response to receiving a user input to change a rotational position of the shaft of the surgical instrument.

Example 6 is a device as in any of Examples 1-5, wherein the first helical gear and the second helical gear are simultaneously rotated to cause rotation of the rotor gear, and wherein the rotation of the rotor gear causes rotation of the shaft of the surgical instrument.

Example 7 is a device as in any of Examples 1-6, wherein one or more of the first helical gear or the second helical gear is rotated to cause clockwise rotation of the rotor gear, and wherein the rotation of the rotor gear causes rotation of the shaft of the surgical instrument.

Example 8 is a device as in any of Examples 1-7, wherein one or more of the first helical gear or the second helical gear is rotation to cause counterclockwise rotation of the rotor gear, and wherein the rotation of the rotor gear causes rotation of the shaft of the surgical instrument.

Example 9 is a device as in any of Examples 1-8, wherein a longitudinal axis of the rotor gear is substantially parallel to a longitudinal axis of the shaft; wherein a longitudinal axis of the first helical gear is substantially perpendicular to the longitudinal axis of the rotor gear; and wherein a longitudinal axis of the second helical gear is substantially perpendicular to the longitudinal axis of the rotor gear.

Example 10 is a device as in any of Examples 1-9, wherein each of the first drive shaft and the second drive shaft comprises: a proximal portion comprising an elliptical cross-sectional geometry; a middle portion comprising a hexagonal cross-sectional geometry or an elliptical cross-sectional geometry; and a distal portion comprising an elliptical cross-sectional geometry; wherein the middle portion is disposed between the proximal portion and the distal portion; and wherein the proximal portion is proximal to the tool receptacle relative to the distal portion.

Example 11 is a device as in any of Examples 1-10, wherein at least a portion of each of the first drive shaft and the second drive shaft comprises a hexagonal cross-sectional geometry; wherein each of the first bore and the second bore is defined by a plurality of sidewalls defining a hexagonal cross-sectional geometry; wherein the first drive shaft is disposed within the first bore such that the first drive shaft and the plurality of sidewalls of the first bore form one or more of a slip fit or an interference fit; and wherein the second drive shaft is disposed within the second bore such that the second drive shaft and the plurality of sidewalls of the second bore form one or more of a slip fit or an interference fit.

Example 12 is a device as in any of Examples 1-11, wherein the surgical instrument is an endoscopic visualization system, and wherein the shaft of the surgical instrument is a rigid laparoscope configured for insertion into a body.

Example 13 is a device as in any of Examples 1-12, wherein the tool drive adaptor further comprises a magnet; and wherein the tool receptacle of the robotic surgical system further comprises a Hall effect sensor.

Example 14 is a device as in any of Examples 1-13, wherein the Hall effect sensor provides an electronic output in response to the magnet of the tool drive adaptor coming within a threshold distance of the Hall effect sensor; and wherein the electronic output indicates that the tool drive adaptor is likely fully installed within the tool receptacle.

Example 15 is a device as in any of Examples 1-14, further comprising an equipment communication subsystem, wherein the equipment communication subsystem comprises memory storing: an identification of the surgical instrument; a serial number of the surgical instrument; and a configuration file for robotically controlling the surgical instrument.

Example 16 is a device as in any of Examples 1-15, wherein the surgical instrument is an endoscopic visualization instrument, and wherein the surgical instrument comprises a cable comprising: a fiber optic bundle for transmitting electromagnetic radiation from an emitter to a distal end of the shaft; and an electrically conductive cable for bidirectionally transmitting data.

Example 17 is a device as in any of Examples 1-16, wherein the surgical instrument is an endoscopic visualization instrument, and wherein the surgical instrument comprises: an image sensor disposed at a distal end of the shaft; and a microcontroller, wherein the microcontroller is in communication with the image sensor, and wherein a controller of the robotic surgical system is in communication with the microcontroller.

Example 18 is a device as in any of Examples 1-17, further comprising: a chassis, wherein the first drive shaft is disposed through a first hole through the chassis, and wherein the second drive shaft is disposed through a second hold through the chassis; and an upper housing configured to attach to the chassis; wherein the upper housing is removable.

Example 19 is a device as in any of Examples 1-18, wherein a controller of the robotic surgical system executes a motion control algorithm to synchronously drive rotation of the first drive shaft and the second drive shaft; and wherein synchronous rotation of the first drive shaft and the second drive shaft causes the shaft to rotate clockwise or counterclockwise about a longitudinal axis of the shaft.

Example 20 is a device as in any of Examples 1-19, further comprising a latching system for unlocking the device from the tool receptacle of the robotic surgical system, wherein the latching system comprises: a latch configured to be depressed inward toward the shaft of the surgical instrument; a latch pin mechanically coupled to the tool receptacle when the device is locked within the tool receptacle; and a latch spring; wherein depression of the latch causes the device to unlock from the tool receptacle.

It will be appreciated that various features disclosed herein provide significant advantages and advancements in the art. The following claims are exemplary of some of those features.

In the foregoing Detailed Description of the Disclosure, various features of the disclosure are grouped together in a single embodiment for the purpose of streamlining the disclosure. This method of disclosure is not to be interpreted as reflecting an intention that the claimed disclosure requires more features than are expressly recited in each claim. Rather, inventive aspects lie in less than all features of a single foregoing disclosed embodiment.

It is to be understood that any features of the above-described arrangements, examples, and embodiments may be combined in a single embodiment comprising a combination of features taken from any of the disclosed arrangements, examples, and embodiments.

It is to be understood that the above-described arrangements are only illustrative of the application of the principles of the disclosure. Numerous modifications and alternative arrangements may be devised by those skilled in the art without departing from the spirit and scope of the disclosure and the appended claims are intended to cover such modifications and arrangements.

Thus, while the disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

The foregoing description has been presented for the purposes of illustration and description. It is not intended to be exhaustive or to limit the disclosure to the precise form disclosed. Many modifications and variations are possible in light of the above teaching. Further, it should be noted that any or all the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. A device for controlling rotational movement of a surgical instrument, the device comprising:
a rotor gear attached to a shaft of the surgical instrument;
a first helical gear rotatably coupled to the rotor gear, wherein the first helical gear comprises a first bore;
a first drive shaft disposed through the first bore of the first helical gear;
a second helical gear rotatably coupled to the rotor gear, wherein the second helical gear comprises a second bore; and
a second drive shaft disposed through the second bore of the second helical gear.

2. The device of claim 1, wherein the device is a tool drive adaptor that is mechanically coupled to a tool receptacle of a robotic surgical system to enable the robotic surgical system to control the rotational movement of the surgical instrument.

3. The device of claim 2, further comprising:
a first input puck attached to the first drive shaft; and
a second input puck attached to the second drive shaft;
wherein a first motor of the tool receptacle is mechanically coupled to the first input puck to drive rotation of the first drive shaft; and
wherein a second motor of the tool receptacle is mechanically coupled to the second input puck to drive rotation of the second drive shaft.

4. The device of claim 3, wherein a controller of the robotic surgical system provides instructions to a robotic arm of the robotic surgical system to actuate one or more of the first motor or the second motor of the tool receptacle; and
wherein the controller of the robotic surgical system provides the instructions to actuate the one or more of the first motor or the second motor in response to receiving a user input to change a rotational position of the shaft of the surgical instrument.

5. The device of claim 1, wherein the first helical gear and the second helical gear are simultaneously rotated to cause rotation of the rotor gear, and wherein the rotation of the rotor gear causes rotation of the shaft of the surgical instrument.

6. The device of claim 1, wherein one or more of the first helical gear or the second helical gear is rotated to cause clockwise rotation of the rotor gear, and wherein the rotation of the rotor gear causes rotation of the shaft of the surgical instrument.

7. The device of claim 1, wherein one or more of the first helical gear or the second helical gear is rotation to cause counterclockwise rotation of the rotor gear, and wherein the rotation of the rotor gear causes rotation of the shaft of the surgical instrument.

8. The device of claim 1, wherein a longitudinal axis of the rotor gear is substantially parallel to a longitudinal axis of the shaft;
wherein a longitudinal axis of the first helical gear is substantially perpendicular to the longitudinal axis of the rotor gear; and
wherein a longitudinal axis of the second helical gear is substantially perpendicular to the longitudinal axis of the rotor gear.

9. The device of claim 2, wherein each of the first drive shaft and the second drive shaft comprises:
a proximal portion comprising an elliptical cross-sectional geometry;
a middle portion comprising a hexagonal cross-sectional geometry; and
a distal portion comprising an elliptical cross-sectional geometry;
wherein the middle portion is disposed between the proximal portion and the distal portion; and
wherein the proximal portion is proximal to the tool receptacle relative to the distal portion.

10. The device of claim 9, wherein the middle portion of the first drive shaft is keyed to a geometry of the first bore such that the first drive shaft transfers rotational movement to the first helical gear; and
wherein the middle portion of the second drive shaft is keyed to a geometry of the second bore such that the second drive shaft transfers rotational movement to the second helical gear.

11. The device of claim 1, wherein at least a portion of each of the first drive shaft and the second drive shaft comprises a hexagonal cross-sectional geometry;
wherein each of the first bore and the second bore is defined by a plurality of sidewalls defining a hexagonal cross-sectional geometry;
wherein the first drive shaft is disposed within the first bore such that the first drive shaft and the plurality of sidewalls of the first bore form one or more of a slip fit or an interference fit; and
wherein the second drive shaft is disposed within the second bore such that the second drive shaft and the plurality of sidewalls of the second bore form one or more of a slip fit or an interference fit.

12. The device of claim 1, wherein the surgical instrument is an endoscopic visualization system, and wherein the shaft of the surgical instrument is a rigid laparoscope configured for insertion into a body.

13. The device of claim 2, wherein the tool drive adaptor further comprises a magnet; and
wherein the tool receptacle of the robotic surgical system further comprises a Hall effect sensor.

14. The device of claim 13, wherein the Hall effect sensor provides an electronic output in response to the magnet of the tool drive adaptor coming within a threshold distance of the Hall effect sensor; and
wherein the electronic output indicates that the tool drive adaptor is likely fully installed within the tool receptacle.

15. The device of claim 1, further comprising an equipment communication subsystem, wherein the equipment communication subsystem comprises memory storing:
an identification of the surgical instrument;
a serial number of the surgical instrument; and
a configuration file for robotically controlling the surgical instrument.

16. The device of claim 1, wherein the surgical instrument is an endoscopic visualization instrument, and wherein the surgical instrument comprises a cable comprising:
a fiber optic bundle for transmitting electromagnetic radiation from an emitter to a distal end of the shaft; and
an electrically conductive cable for bidirectionally transmitting data.

17. The device of claim 2, wherein the surgical instrument is an endoscopic visualization instrument, and wherein the surgical instrument comprises:
an image sensor disposed at a distal end of the shaft; and
a microcontroller, wherein the microcontroller is in communication with the image sensor, and wherein a controller of the robotic surgical system is in communication with the microcontroller.

18. The device of claim 1, further comprising:
a chassis, wherein the first drive shaft is disposed through a first hole through the chassis, and wherein the second drive shaft is disposed through a second hold through the chassis; and
an upper housing configured to attach to the chassis;
wherein the upper housing is removable.

19. The device of claim 2, wherein a controller of the robotic surgical system executes a motion control algorithm to synchronously drive rotation of the first drive shaft and the second drive shaft; and
wherein synchronous rotation of the first drive shaft and the second drive shaft causes the shaft to rotate clockwise or counterclockwise about a longitudinal axis of the shaft.

20. The device of claim 2, further comprising a latching system for unlocking the device from the tool receptacle of the robotic surgical system, wherein the latching system comprises:
a latch configured to be depressed inward toward the shaft of the surgical instrument;
a latch pin mechanically coupled to the tool receptacle when the device is locked within the tool receptacle; and
a latch spring;
wherein depression of the latch causes the device to unlock from the tool receptacle.
